(19) United States
(12) Patent Application Publication
(10) Pub. No.: US 2019/0065909 A1
(43) Pub. Date: Feb. 28, 2019

(54) METHOD FOR EVALUATING A CHEWING FUNCTION TEST

(71) Applicant: bredent medical GmbH & Co. KG, Senden (DE)

(72) Inventor: Gregor SLAVICEK, Stuttgart-Birkach (DE)

(73) Assignee: bredent medical GmbH & Co. KG, Senden (DE)

(21) Appl. No.: 16/170,261

(22) Filed: Oct. 25, 2018

Related U.S. Application Data
(63) Continuation of application No. PCT/EP2017/059662, filed on Apr. 24, 2017.

(30) Foreign Application Priority Data
Apr. 26, 2016 (DE) 10 2016 107 689.9

(51) Int. Cl. G06K 9/62; A61B 5/11; A61B 5/00; G06T 7/90; A23L 29/281; A23L 5/43

(52) U.S. Cl. CPC G06K 9/6267; A61B 5/1114; A61B 5/0077; G06T 7/90; A61B 5/1128; A23V 2002/00; A23L 29/281; A23L 5/43; G06T 2207/30242; G06T 2207/10028; G06T 2207/10024; A61B 5/4542

(57) ABSTRACT

A method for evaluation of a chewing function test includes the following steps. Spit-out chewed model food with chewing function pieces is made available. The chewed model food is collected in a sieve, and subsequently rinsing of the chewed model food is carried out, so as to obtain saliva-free unchewed chewing function pieces or saliva-free particles of the chewing function pieces, which particles include chewed chewing function pieces. Afterward, separation of the particles takes place. After a determination of the total number of particles, classification of the total number of particles takes place using predetermined standard values, which classification includes a differentiation with regard to unchewed components of the model food, partly chewed components without split-off particles of the model food, and split-off particles.

### CROSS REFERENCE TO RELATED APPLICATIONS

This application is a continuation of and Applicant claims priority under 35 U.S.C. § 120 of International Application No. PCT/EP2017/059662 filed on Apr. 24, 2017 which claims priority under 35 U.S.C. 119 of German Application No. 10 2016 107 689.9 filed on Apr. 26, 2016. The international application under PCT article 21(2) was not published in English. The disclosures of the aforesaid International Application and German application are incorporated by reference.

### BACKGROUND OF THE INVENTION

### 1. Field of the Invention

The invention relates to a method for evaluation of a chewing function test.

### 2. Description of the Related Art

From the general state of the art, it is known to analyze the chewing process of a human being by means of the use of elastic model food. In such studies, a test subject is given such model food as a chewing sample, which is to be chewed for a certain period of time on one or both sides of the jaw, without swallowing any particles of the model food that split off. After completion of the chewing test, the chewed material is spit into a sieve by the test subject, and can subsequently be analyzed.

The result of such a chewing function test, however, depends on many different factors, such as, for example, the condition of the chewing surfaces, the presence of dental prostheses or the like. Now in order to be able, within the scope of larger studies, not only to carry out comprehensive chewing function test series but also to evaluate individual chewing function tests as efficiently as possible, comprehensive work effort is therefore required on the part of the persons involved in the study, whereby possibly, study results might be interpreted incorrectly or inaccurately due to non-standardized work procedures.

In the technical article by Zenginel et al. (Nutritional-Blood-Markers And Dental Status, Int. Poster J Dent Oral Med, Vol. 14, No. 4, 625, 2012), the focus of an exploratory study was placed on the connection between tooth status and some nutrition-related blood parameters, wherein the chewing test on the basis of carrots was used only as an additional test.

In the article by Mahmood et al. (Use of image analysis to determine masticatory efficiency in patients presenting for immediate dentures, International Journal of Prosthodontics, Quintessence Publ., Vol. 5, No. 4, 1992), an optical method is presented, which measures the surface area, the length, and the width of the comminuted particles. Cylindrical carrot pieces (20 mm×6 mm) are used as the test medium. The comminuted particles are collected in a Petri dish after a specific number of chewing cycles or a complete chewing sequence, and separated. The test subjects either chewed for 20 cycles or carried out a complete chewing sequence. Different stages of a prosthetic treatment (full denture) were compared with test subjects having natural teeth and test subjects having full dentures.

In the article by Van der Bilt et al. (A comparison between sieving and optical scanning for the determination of particle size distribution obtained by mastication in man, Archives Of Oral Biology, Vol. 38, No. 2, Page 159-162, 1993), an experimental study for comparison of the sieving method with an optical method (flat-bed scanner) for evaluation of a mixture of particles having different sizes, as they occur during chewing, is presented. Pre-finished particles having a specific size are chewed (20, 40, and 80 seconds). The surface area of the particles is calculated by way of the shortest and the longest diameter. Subsequently, a conclusion is drawn regarding the volume of the individual particles, by way of specific parameters. The test medium used is Optosil (silicone). For the optical scanning method, a commercially available scanner is used, wherein overly small particles are still eliminated before the scan. The surface area of the individual particles is measured by measuring the longest and widest distance. In addition, many measurements of the diameter of the particles along a plurality of directions are carried out. The sieving method is fundamentally based on the volume of the particles. Therefore it is necessary, in this method of procedure, to estimate the volume of the particles. This estimate is done by way of specific assumptions, wherein a specific shape of the chewed particles is assumed.

In the article by Sugimoto et al. (New Image analysis of large food particles can discriminate experimentally suppressed mastication, Journal Of Oral Rehabilitation, Vol. 39, No. 6, 2012), an analysis method on the basis of a dark-field analysis is presented, in order to determine the chewing efficiency in a specific patient population, i.e. older people with swallowing problems. In this regard, the focus lies on the analysis of larger chewed particles having a diameter greater than 2 mm. In this regard, the chewing efficiency is tested using different natural foods. Restriction of the chewing performance took place by way of monitoring of the electromyography (EMG) conductance of the chewing muscle (musculus masseter). The focus of this method lies on recognizing larger particles, which, however, represent a significant risk (infection of the airways, reaching to pneumonia, occlusion of the airways reaching to suffocation) for the population in question (swallowing problems with the increased possibility of aspiration of food components).

A further method described in US 2013/222560 A1 filters specific light frequencies multiple times, using microscopy in the dark field, in complicated manner, for evaluation of the chewing samples. The comminuted particles are prepared in multiple steps, i.e. compressed and embedded between two plates. For this purpose, the particles are first pressed through a filter, and also positioned.

In DE 11 2005 002 627 T5, an apparatus for determination of the chewing efficiency is described, in which apparatus chewed pieces are introduced into a stirring fluid. In this regard, a rubber gel is used, for example. Here, the increased extraction of specific components of the food after comminution by means of the chewing process is used to determine the chewing efficiency.

In US 2005/220713 A1, a method is described, in which a chewing sample composed of wax strips dyed in different colors is used, which strips do not break down during the mechanical stress during the chewing process. The individual wax strips, dyed in different colors, are mixed during this process, so that a conclusion regarding the chewing efficiency can be drawn on the basis of the mixed colors.

On the basis of the aforementioned prior art, however, it must be stated that carrots can hardly be standardized as a starting material for chewing test methods, due to their variety of types and their different storage conditions. If silicone is used as a test medium, this test medium again does not correspond to a natural food, wherein here, it is less the elastic/plastic properties but rather the negative influence on the natural chewing processes that are relevant. The test subject will involuntarily try to avoid swallowing the artificial pieces, and therefore will change the muscle balance during the chewing process. When using plastic strips in the mixed method described above, a significant element during chewing natural foods, during division of the food bolus into ever smaller pieces, is lost. Accordingly, it becomes increasingly difficult to position the many small pieces, which are held together by the saliva, back between the rows of teeth. For this reason, such mixed methods have only limited informational value for the actual chewing efficiency.

There is therefore a need to create a method that allows a reliable statement regarding the chewing function and, beyond that, an automatic evaluation of a chewing function test.

### SUMMARY OF THE INVENTION

This task is accomplished by means of the characteristics of the method according to the invention. Further advantageous embodiments of the invention are discussed below. These embodiments can be combined with one another in technologically practical manner. The description, in particular in connection with the drawing, additionally characterizes and illustrates the invention.

According to the invention, a method for evaluation of a chewing function test is indicated, which comprises the following steps. Spit-out chewed model food with chewing function pieces is made available. The chewed model food is collected in a sieve, and subsequently rinsing of the chewed model food is carried out, in order to obtain saliva-free unchewed chewing function pieces or saliva-free particles of the chewing function pieces, which comprise chewed chewing function pieces. Afterward, separation of the particles takes place. After determination of the total number of particles, classification of the total number of particles using predetermined standard values takes place; this classification comprises a differentiation with regard to unchewed components of the model food, partially chewed components without split-off particles of the model food, and split-off particles.

Accordingly, a method is created, which starts with making available spit-out chewed model food with chewing function pieces. In this regard, as mentioned initially, the actual chewing process for chewing the model food can be carried out within established time spans and with adherence to prescribed sequences, such as, for example, chewing on only one side of the jaw or the like. The chewed model food is subsequently rinsed, so as to obtain either the unchewed chewing function pieces or the chewed chewing function pieces, depending on the chewing ability of the test subject, in the form of particles, which are freed of wetting with saliva by means of the rinsing process.

In this regard, for example, the chewed model food can be spit into a sieve, which is subsequently rinsed under cold water. Subsequently, separation of the particles takes place, so that these particles are isolated from one another, wherein for this purpose, a pre-finished paper sheet can preferably be used as a recording sheet. Subsequently, the total number of particles is determined, wherein the total number of particles is classified, in conclusion, in that the total number obtained is compared with previously determined standard values, which can also take the individual state of the test subject into consideration. In this regard, classification can be undertaken using a scale that follows the known school grade system, for example. Accordingly, a method for evaluation of the chewing function test, which method is simple but also reproducible, due to the standardized method of procedure, is obtained, which method can be carried out without any great effort, using the simple classification rules, and accordingly permits almost no incorrect conclusions in the interpretation of the test results.

A fruit gum compound with different gelatin proportions to adjust the degree of hardness is clearly superior to the foods mentioned initially, such as carrots, cooked meat, and peanuts, which have the problems of lack of standardization as already mentioned several times.

In this regard, the test subjects do more than simply carry out a predetermined number of chewing cycles, so that the result shows more than just overall efficiency. The invention also permits differentiated evaluation, so that all the chewing sequences, chewing sequences on the right side, left side, and both sides, chewing sequences for soft, medium, and hard can be included. In particular, no random selection of the chewing side by the test subjects takes place.

In contrast to collection and separation of the chewing samples in a Petri dish filled with water, according to the invention collection of the chewed particles takes place in a sieve, which allows removal not only of saliva but also of air bubbles under running water. The particles are applied to a recording sheet in a state in which they are still damp but not watery, so that clear separation is possible. The recording sheet has a standardized recording surface area of 17.5 cm×11 cm (192.5 cm²). The distortion effect that occurs in every optical survey can be monitored by means of reference points, e.g. circles having a diameter of precisely 1 cm. Before the surface areas are determined for the individual pieces (by way of the actual surface areas and not by way of calculation of the surface area by way of the length and width of the chewed particles), the optical distortion is corrected.

The evaluation of chewing efficiency takes place by way of the parameters number of particles, surface area of the particles, and distribution of the surface areas. A comparison with standard values, which are individually adapted on the basis of demographic data, is also essential.

The evaluation of chewing efficiency therefore relates not simply to the overall efficiency (result of a chewing sequence, wherein the test subject can randomly choose the chewing side), but rather is based on a detailed analysis per side, per degree of hardness, per chewing sequence.

The preparation of the samples required for the dark-field method, as described above (i.e. washing, rinsing, and sieving), and the introduction into a medium (benzalkonium chloride) differs significantly from the invention, in which rinsing in a screen under running water represents the sole manipulation of the chewing samples. Likewise, in the dark-field method, only the outline is determined; two-dimensional or three-dimensional measurement by means of optical sensors does not take place.

According to an embodiment of the invention, the predetermined standard values are individualized with regard to the patient, for classification.

According to a further embodiment of the invention, an optical measurement of the particles is carried out. In this regard, the optical measurement can be carried out two-dimensionally or three-dimensionally.

Accordingly, alternatively or additionally, not only is the total number of particles or the distinction between unchewed components of the model food, partially chewed components without split-off particles of the model food, and split-off particles determined, but in addition, a geometric survey of the particles is also undertaken. Such an optical survey thereby increases the informational content of the chewing function test, wherein the geometric dimensions that are determined can also be processed further in automated manner. When using multiple light sensors for optical detection, a three-dimensional optical survey can also be achieved.

According to a further embodiment of the invention, the size of the particles, their volume and number are compared with data of a database that represent standard data. In this regard, the standard data of the data base can be individualized for a test subject. Preferably, an age of the test subject, a tooth status or a prosthetic status are taken into consideration.

An automated evaluation can particularly take place in that the size of the particles, their volume and number are compared with standard values from a database. In this regard, it is provided, once again, that the standard data of the database are individualized with regard to a test subject, so that an age of the test subject, a tooth status or a prosthetic status, for example, can be taken into consideration. The individualized standard data therefore indicate how a test subject with the corresponding biological data should be able to chew. By means of a comparison of an individual test subject with such individualized standard data, a deviation of the chewing function can therefore be reliably determined.

According to a further embodiment of the invention, lower jaw movements during chewing of the model food are additionally recorded. In this regard, lower jaw movements can be recorded with a camera or a device for joint path recording.

In addition to the analysis of the chewed model food, it is provided to record the lower jaw movements that occur during chewing of the model food. This recordation can be done using a camera, for example. Thereby both the extent of the chewing movement and the position of the chewing movements in comparison with standard movements such as protrusion, retrusion or opening and closing or mediotrusion on both sides of the jaw can be analyzed. For this purpose, every chewing movement should be started from a reference position, so as to be able to evaluate the position of the chewing movement with regard to this reference position. Such analyses can be undertaken on both sides of the jaw.

According to a further embodiment of the invention, the chewing function pieces are produced from a gelatin compound, as cylindrical bodies, and the model food comprises chewing function pieces having different hardness. The model food with chewing function pieces having different hardness can be chewed by a patient in a chewing sample. In this regard, the step of making the chewed model food available can comprise different chewing positions in the mouth of a patient.

Model food is typically produced in cylindrical shape, with a known degree of hardness and standardized outside dimensions. The model food that is to be used for analysis of the chewing is elastic and can be based on a conventional gelatin compound, such as that used for production of commercially available fruit gum. It has a cylindrical standard shape, which typically can have a height of 1 cm and a diameter of 2 cm, and is produced in three different degrees of hardness (soft, medium, hard). The different degrees of hardness are achieved by addition of different amounts of gelatin to the basic compound (soft: 15.5 g per compound; medium: 23 g per compound; hard: 31 g per compound). The different degrees of hardness can be dyed with different natural dyes (hard: red—elderberry, medium: yellow—turmeric, soft: green—chlorophyll) and provided with strawberry flavor at the same intensity. It is advantageous that as a result, optical measurement or classification or both are facilitated.

Furthermore, a processor is indicated, which receives commands from a memory, which commands are suitable for carrying out a method described above.

The processor can be a component of a mobile telephone or tablet computer, wherein the commands are made available in the form of an application software for mobile devices, wherein a camera of the mobile telephone or tablet computer can be controlled for an optical survey of the particles.

The processor can be a component of a data processing system that is coupled with a light sensor or a camera for optical measurement of the particles. The camera can be equipped with two filters, which can filter out reflections. The filters are disposed at an angle of 90° relative to one another, for example. In this regard, one filter is situated in front of a flash; the other filter is situated in front of a lens of the camera. In this way, two-dimensional evaluation, in particular, can take place more precisely.

Finally, a software program product is indicated, which contains commands that are readable for a processor and are suitable for implementing a method described above.

In this regard, the automatic evaluation of the particles can take place using a suitable camera. In a preferred embodiment, both the camera and also the software required for the evaluation are components of a mobile telephone, so that such an evaluation of a chewing function test according to the method according to the invention can also be carried out in automated manner, without support from clinic personnel. In order to achieve this evaluation with as much informational value as possible, it can also be provided, aside from simple classification, that the software is made available in the form of an application software for mobile devices, wherein a user only needs to enter any individual information required for evaluation one time.

### BRIEF DESCRIPTION OF THE DRAWINGS

In the following, some exemplary embodiments will be explained in greater detail using the drawings. In the drawings:

FIG. 1 shows the schematic sequence of the method according to the invention according to one embodiment, and

FIG. 2 shows schematically, an example of model food in chewed form when using the method according to the invention, and

FIG. 3 shows a structure for carrying out the method according to the invention, according to one embodiment.

### DETAILED DESCRIPTION OF THE PREFERRED EMBODIMENTS

In the figures, components that are the same or have the same functional effect are provided with the same reference symbols.

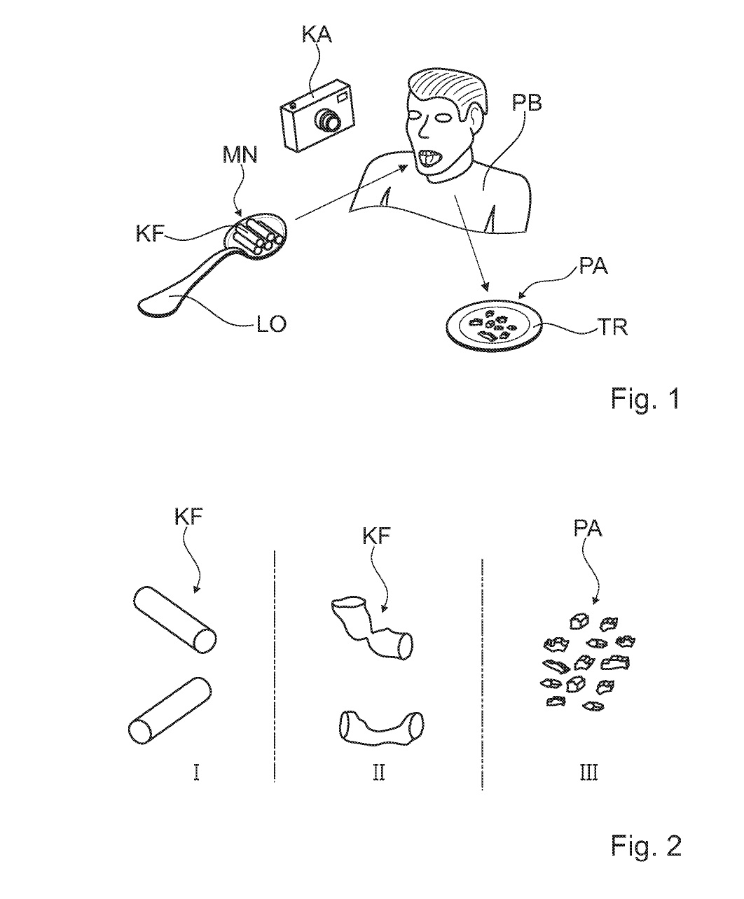

In FIG. 1, the schematic sequence of the method according to the invention is shown. Accordingly, model food MN with chewing function pieces KF is made available to a test subject PB as cylindrical bodies composed of a gelatin compound, for example by means of a spoon LO. The model food MN is produced in cylindrical shape with different degrees of hardness and standardized outside dimensions. The model food MN that is to be used for analysis of chewing is elastic and is based on a conventional gelatin compound, as it is used for the production of commercially available fruit gum. This model food has a cylindrical standard shape, which typically can have a height of 1 cm and a diameter of 2 cm, and is produced in three different degrees of hardness (soft, medium, hard). The different degrees of hardness are achieved by addition of different amounts of gelatin to the base compound (e.g. soft: 15.5 g per compound; medium: 23 g per compound; hard: 31 g per compound). The different degrees of hardness can be dyed with different natural dyes, and provided with flavors having the same intensity.

The test subjects are asked to sit down in the dentist's chair. It is explained to them that they should first comprehensively apply saliva to the model food MN on the right or left side, to the following chewing sample, then to chew it at maximal chewing intensity, for example for 30 seconds, without swallowing any particles. At the end of the chewing phase, a signal for stopping sounds. The test subject now spits out what has been chewed, as shown on the right in FIG. 1.

The pieces covered with saliva are rinsed under running cold water and thereby freed of saliva.

The method according to the invention begins with making available spit-out chewed model food MN with chewing function pieces KF. In this regard, as has been mentioned, the actual chewing process for chewing the model food MN can be carried out with predetermined time spans and with adherence to prescribed procedures, such as chewing on only one side of the jaw or the like, for example. All the particles that are in the mouth are collected. The chewed model food MN is subsequently rinsed, so as to obtain either the unchewed chewing function pieces or the chewed chewing function pieces in the form of particles PA, depending on the chewing ability of the test subject; these particles are freed of wetting with saliva by means of the rinsing process.

For this purpose, the chewed model food MN can be spit into a sieve (not shown in FIG. 1), for example, which sieve is subsequently rinsed under cold water.

The sieve is emptied onto a pre-finished paper sheet. Subsequently, separation of the particles takes place, so that these are isolated from one another, wherein for this purpose, the pre-finished paper sheet can preferably be used as a carrier TR. For this purpose, the particles are sorted out from one another within the area in question, using two spatulas, and isolated from one another.

An example of chewed model food is shown in FIG. 2. Depending on the chewing function of the test subject, the model food MN is now present with unchewed components of the model food MN as in region I, partially chewed components without split-off particles of the model food MN as in region II or with split-off particles PA of the model food MN as in region III.

Subsequently, the total number of particles PA is determined, wherein the total number of particles PA is conclusively classified, in that the total number obtained is compared with previously determined standard values, which can also take the individual condition of the test subject into consideration. In this regard, the classification can be undertaken using a scale that follows the known school marking system, for example. Accordingly, a method for evaluation of the chewing function test, which method is simple but reproducible due to the standardized method of procedure, is obtained, and it can be carried out without greater effort due to the simple classification rules, and accordingly allows almost no incorrect conclusions in the interpretation of the test results.

All the particles PA are counted for classification, wherein this takes place independent of the size of each individual particle PA. Solely the quantity is evaluated and subdivided into categories, which correspond to the number of particles. For this purpose, the following break-down can be selected: 0=no data collected, 1>20 particles, 2>10 particles, 3>5 particles, 4>1 particle, 5=1 particle, 6=unchewed. Large, cohesive particles are counted as 1, and Category 5 also is used for a preserved or only partially chewed chewing sample, which did not, however, contain any completely split-off particles.

For study purposes or documentation purposes, the particles can be dried until they are dry and firmly seated on the paper sheet. The sheet of paper can be placed in a clear plastic sleeve and archived in a file folder.

A complete chewing function test is composed of a total of 9 chewing sequences: the respective hardness of the model food is chewed once on the right side, then on the left side, and finally on both sides. This test is done in a predetermined sequence:
Step 1 right side/soft model food,
Step 2 left side/soft model food,
Step 3 both sides/soft model food,
Step 4 right side/medium model food,
Step 5 left side/medium model food,
Step 6 both sides/medium model food,
Step 7 right side/hard model food,
Step 8 left side/hard model food, and
Step 9 both sides/hard model food.

In addition, lower jaw movements during chewing of the model food MN can be recorded. In addition to the analysis of the chewed model food MN as just described, it is provided to record the lower jaw movements that occur during chewing of the model food MN. This can be done using a camera KA or a device for joint path recording, for example. Therefore both the extent of the chewing movement and the position of the chewing movement can be analyzed in comparison with standard movements such as protrusion, retrusion or opening and closing, i.e. mediotrusion on both sides of the jaw of the test subject. For this purpose, every jaw movement should be started from a reference position, so as to be able to evaluate the position of the chewing movement with regard to this reference position. Such analyses can be undertaken on both sides of the jaw.

The test bodies that the test subject comminutes in a standardized chewing sequence, as model food MN, are standardized in terms of size and shape, have elastic properties (no nuts or carrots, which can be ground by forces; in contrast, the elastic properties represent a great challenge for the tooth surfaces of occlusion, with cusps that demonstrate cutting functions, and become increasingly smaller (in contrast to test methods based on chewing gum). Accordingly, it becomes more difficult that pieces of the model food MN are positioned between the teeth. Thereby the chewing efficiency in its totality is represented as an activity of the chewing muscles, of the efficiency of occlusion, and of the coordination of tongue and cheek, so as to reposition the food.

The entire test method does not simply consist of a chewing sequence, but rather takes into consideration the great adaptation possibility of the chewing organ: if no teeth are present on one side, then a one-time chewing function test can certainly represent very good but only one-sided chewing efficiency. Therefore a false negative result of a one-sided chewing test could certainly be obtained.

The chewing organ is not put under such great stress by a one-time chewing test with a duration of 30 sec or even 60 sec that latent problems can already be recognized. For this reason, the chewing function test is based on multiple degrees of hardness (soft, medium, and hard) and a total of 9 tests.

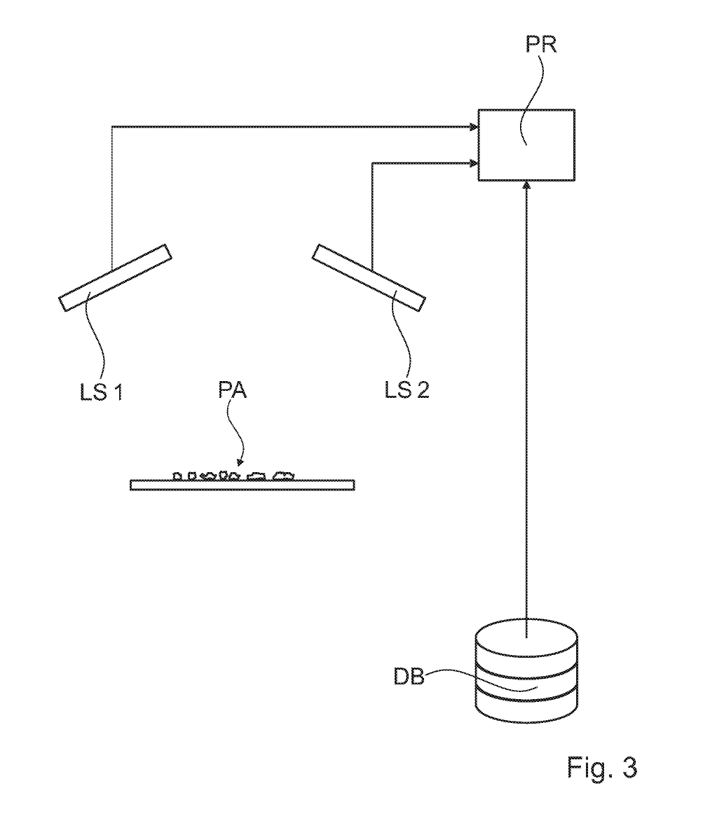

Making reference to FIG. 3, a variant of the method for evaluation of the chewing function test is described, which can take place essentially in automated manner.

For this purpose, an optical survey of the particles by means of a light sensor LS1 is additionally carried out. Therefore the optical survey can be carried out in two dimensions. In order to be able to conduct a three-dimensional survey, at least one further light sensor LS2 is provided. A charge-coupled device (CCD) sensor, for example, can be used as a light sensor. It is also possible to make additional light sensors available so as to further increase the processing precision. Furthermore, however, it is also conceivable to use a laser scanner for a two-dimensional or three-dimensional optical survey.

To improve the quality of the optical survey, two filters can be provided, which can filter out reflections. The filters are disposed at an angle of 90° relative to one another, for example. In this regard, one filter is situated in front of a flash; the other filter is situated in front of a lens of the CCD sensor. In this way, a two-dimensional evaluation, in particular, can take place more precisely.

Accordingly, alternatively or additionally, not only is the total number of particles PA or the distinction between unchewed components of the model food, partially chewed components without split-off particles of the model food, and split-off particles determined, but in addition, a geometrical dimension such as size or volume of the particles is determined.

In this regard, classification can be undertaken using a scale that takes the size distribution of the chewed pieces of the model food into consideration. Such an optical survey by means of the light sensors LS1 and LS2 thereby increases the informational value of the chewing function test, wherein the images obtained can be processed further to determine the geometrical dimensions, by means of a processor PR.

In this regard, the processor can be connected with a database DB, so as to compare the size of the particles, their volume and quantity with data of the database DB that represent standard values. Furthermore, the standard data of the database DB can be individualized with regard to a test subject, wherein preferably, an age of the test subject, a tooth status or a prosthetic status is/are taken into consideration. The individualized standard data thereby indicate how a test subject with the corresponding biological data should be able to chew. By means of a comparison of an individual test subject with such individualized standard data, a deviation of the chewing function can thereby be reliably determined.

The processor PR can be a component of a mobile telephone or tablet computer (not shown in FIG. 3), wherein the method is made available in the form of an application software for mobile devices. A camera of the mobile telephone or tablet computer can be turned on as a light sensor LS1 for optical measurement of the particles PA.

Likewise, it is possible that the processor PR is a component of a data processing system that is coupled with a camera as a light sensor LS1 for optical measurement of the particles.

The optical automated evaluation of the particles PA yields not only the quantity or the surface area or the volume of the particles PA, but also the distribution function of the particles PA. Specifically from this distribution function, it can be recognized how efficiently the chewing organ was able to comminute the test body—in other words the total function composed of activity of the chewing muscles, the efficiency of occlusion (or also the quality of the prosthetic treatment), and the coordination of the mimic muscles and tongue.

Collection of the chewed particles PA once again takes place in a sieve, which allows removal of saliva but also of air bubbles under running water. The particles PA are applied to a recording sheet in a state in which they are still damp but not watery, so that clear separation is possible. The recording sheet has a standardized recording surface area of 17.5 cm×11 cm (192.5 cm²). The distortion effect that occurs during every optical survey is monitored on the recording sheet by means of reference points or reference objects, e.g. circles having a diameter of precisely 1 cm. Before the surface areas are determined for the individual particles PA, which is done by way of the actual surface areas and not by way of calculation of the surface area by way of the length and width of the chewed particles, the optical distortion is corrected.

The optical evaluated evaluation furthermore yields a comparison with an individualized standard, by way of taking into consideration the demographic data such as age, gender, tooth status, prosthetic status, and health status. The dentist is thereby given the opportunity to evaluate the chewing efficiency of the individual patient individually, with reference to his/her representative age group and taking his/her status into consideration. Furthermore, the (optimal) desired status is indicated, which represents the chewing efficiency if the intraoral situation is improved and prosthetically rehabilitated.

The optical automated evaluation takes the following intra-individual comparison possibilities into consideration:
1) right-sided vs. left-sided vs. both-sided chewing, each for soft, medium, and hard,
2) chewing on the right, soft vs. medium vs. hard,
3) chewing on the left, soft vs. medium vs. hard,
4) chewing on both sides, soft vs. medium vs. hard,
5) initial vs. late chewing sequences (since the chewing test is carried out in a standardized sequence, increasing stresses can be recognized from the differences in the initial, intermediate, and late chewing sequences).

The characteristics indicated above and in the claims, as well as the characteristics that can be derived from the figures, can be advantageously implemented both individually and in different combinations. The invention is not restricted to the exemplary embodiments described, but rather can be modified in many different ways, within the scope of the ability of a person skilled in the art.

What is claimed is:

1. A method for evaluating a chewing function test, comprising:
(a) making model food available, the model food comprising chewing function pieces composed of edible material;
(b) chewing the model food at a plurality of predetermined mouth positions to produce sequentially in at least one chewing sample chewed model food with the chewing function pieces;
(c) rinsing the chewed model food to obtain saliva-free unchewed chewing function pieces or saliva-free particles of the chewing function pieces, which comprise chewed chewing function pieces;
(d) separating the saliva-free particles on a recording sheet;
(e) conducting a determining step by determining a total number of particles on the recording sheet;
(f) conducting a classification step by classifying the total number of particles using predetermined standardized values to differentiate with regard to unchewed components of the model food, partially chewed components without split-off particles of the model food, and split-off particles; and
(g) evaluating chewing function based on the classification step.

2. The method according to claim 1, wherein the predetermined standardized values used for classification are individualized with regard to a patient.

3. The method according to claim 1, further comprising carrying out an optical survey of the particles, in two dimensions or three dimensions, to evaluate the chewing function, using the classification step.

4. The method according to claim 3, wherein the optical survey is carried out on the recording sheet, with correction of distortions.

5. The method according to claim 2, wherein size, volume, and number of the particles are compared with standardized data of a database comprising standardized values.

6. The method according to claim 5, wherein the standardized data of the database are individualized for the patient.

7. The method according to claim 6, wherein the standardized data takes into consideration an age of the patient, a tooth status or a prosthetic status.

8. The method according to claim 1, furterh comprising recording lower jaw movements during chewing of the model food.

9. The method according to claim 8, wherein the lower jaw movements are recorded with a camera.

10. The method according to claim 1, wherein the model food is made available with chewing function pieces having different hardness.

11. The method according to claim 10, wherein the model food with chewing function pieces having different hardness is chewed by a patient in one or more chewing samples, which are carried out sequentially.

12. The method according to claim 2, wherein the chewed model food is made available from different chewing positions in a mouth of the patient.

13. The method according to claim 1, wherein the chewing function pieces are produced from a gelatin compound, as cylindrical pieces.

14. A processor that receives commands from a memory, wherein the commands are suitable for carrying out the determining step of determining the total number of particles and the classification step of classifying the total number of particles in the method according to claim 1.

15. A mobile telephone or tablet computer comprising the processor according to claim 14 and applicable software for mobile devices comprising commands to turn on a camera of the mobile telephone or tablet computer for an optical survey of the particles.

16. A data processing system comprising the processor according to claim 14 coupled with a camera for an optical survey of the particles.

17. A software program product, which contains commands that can be read by a processor, wherein the commands are suitable for carrying out the determining step of determining the total number of particles and the classification step of classifying the total number of particles in the method according to claim 1.

18. A model food for carrying out the method according to claim 1, comprising cylindrical bodies having a height of 1 cm and a diameter of 2 cm.

19. The model food according to claim 18, comprising chewable samples with three different degrees of hardness comprising a soft degree of hardness, a medium degree of hardness, and a hard degree of hardness, wherein the different degrees of hardness are achieved by addition of different amounts of gelatin to a base compound.

20. The model food according to claim 19, wherein the chewable samples are is dyed with different natural dyes and provided with flavors having the same intensity.
